(19) United States
(12) Patent Application Publication
(10) Pub. No.: US 2013/0316015 A1
(43) Pub. Date: Nov. 28, 2013

(54) Methods for Repairing Cartilage

(71) Applicants: Rita Kandel, Toronto (CA); Robert Pilliar, Toronto (CA); Marc Grynpas, Toronto (CA); Jean-Philippe St-Pierre, Toronto (CA)

(72) Inventors: Rita Kandel, Toronto (CA); Robert Pilliar, Toronto (CA); Marc Grynpas, Toronto (CA); Jean-Philippe St-Pierre, Toronto (CA)

(73) Assignee: MOUNT SINAI HOSPITAL, Toronto, ON (CA)

(21) Appl. No.: 13/978,501

(22) PCT Filed: Jan. 10, 2012
(86) PCT No.: PCT/CA12/00025

§ 371 Date: Aug. 14, 2013

Related U.S. Application Data
(60) Provisional application No. 61/431,226, filed on Jan. 10, 2011.

(51) Int. Cl. A61K 33/42

(52) U.S. Cl. CPC A61K 33/42
USPC 424/602; 435/375

(57) ABSTRACT

The invention relates to compositions, dosage forms and methods for stimulating cartilage growth, regeneration or repair, and treating cartilage defects and diseases using short chain polyphosphates, in particular polyphosphates having a chain length of greater than 5 phosphate units. The compositions, dosage forms and methods have particular application in the treatment of osteoarthritis.

### FIELD OF THE INVENTION

The invention relates to methods for stimulating cartilage growth, regeneration or repair, and treating cartilage defects and diseases.

### BACKGROUND OF THE INVENTION

Articular cartilage is a connective tissue that covers the articulating surfaces of bones in synovial joints and permits their low friction gliding as well as the transmission and distribution of applied forces to the subchondral bone. The avascular nature of articular cartilage combined with the limited ability of chondrocytes to migrate within the tissue contribute to its limited intrinsic capacity for self-repair following partial thickness damage caused by trauma and/or disease [1,2]. Full thickness defects are temporarily replaced by fibrocartilagenous tissue of inferior mechanical quality due to the disruption of the vascularised subchondral bone [1]. Hence, cartilage tissue damage often leads to the progressive development of osteoarthritis accompanied by pain and loss of range of motion for the patient.

Current surgical methods to enhance the repair and regenerative capabilities of cartilage including surgical marrow stimulation techniques and autologous chondrocyte implantation (ACI) have yielded limited long-term clinical success, partly because of the fibrocartilagenous nature of the resulting repair tissue [1]. Recent efforts to improve on the functional outcome of cell-based techniques such as ACI have resulted in the development of numerous tissue engineering approaches to deliver chondrogenic cells or in vitro-formed hyaline-like cartilage to the injury site. However, tissue engineered cartilage is often characterized by low contents of extracellular matrix components and specifically collagen type II levels compared to native articular cartilage contributing to inferior mechanical properties and limited long-term in vivo survival [3,4].

### SUMMARY OF THE INVENTION

The present invention relates to methods of stimulating or enhancing cartilage repair, growth and/or regeneration in a subject and methods of delivering compounds or pharmaceutical compositions to articular defects to aid in cartilage repair, growth and/or regeneration and to prevent articular degradation.

The present invention also relates to a method of slowing or inhibiting cartilage degradation in a subject, in particular cartilage degradation that occurs in osteoarthritis.

In an aspect, the invention provides a method of stimulating cartilage growth, regeneration or repair in a subject where cartilage growth, regeneration or repair is desired comprising administering to the subject a therapeutically effective amount of a short chain polyphosphate, wherein the short chain polyphosphate has greater than 5 phosphate units

In an aspect, the invention relates to a method of stimulating cartilage growth, regeneration or repair at a site in a subject where cartilage growth, regeneration or repair is desired. The treatment site may be, for example, in a joint (e.g., in the knee, shoulder, ankle, elbow, wrist, fingers and the like). The method comprises the step of administering a therapeutically effective amount of a short chain polyphosphate to a site requiring treatment, in particular a site of injury. In an aspect, the method comprises administering a polyphosphate of greater than about 5 phosphate units to a site requiring treatment. In an embodiment an inorganic polyphosphate of about 10 to 100 phosphate units is administered to a site requiring treatment. In an embodiment an inorganic polyphosphate of about 10 to 85 phosphate units is administered to a site requiring treatment. In an embodiment an inorganic polyphosphate of about 30 to 60 phosphate units is administered to a site requiring treatment. In an embodiment an inorganic polyphosphate of about 40 to 50 phosphate units is administered to a site requiring treatment. In an embodiment an inorganic polyphosphate comprising an average of about 45 phosphate units is administered to a site requiring treatment.

In an aspect, the invention relates to a method of stimulating cartilage growth, regeneration or repair and reducing cartilage mineralization at a site in a subject where cartilage growth, regeneration or repair is desired and mineralization is not desired. The method comprises or consists essentially of the step of administering a therapeutically effective amount of a short chain polyphosphate. In an aspect, the invention relates to a method of restoring or increasing cartilage matrix at a site in a subject where cartilage growth, regeneration or repair is desired comprising or consisting essentially of administering a therapeutically effective amount of a short chain polyphosphate to the site. In an aspect, the invention relates to a method of stimulating extracellular cartilage matrix accumulation at a site in a subject where cartilage growth, regeneration or repair is desired comprising or consisting essentially of administering a therapeutically effective amount of a short chain polyphosphate to the site. In an aspect, the invention relates to a method of restoring or increasing collagen and proteoglycan content at a site in a subject where cartilage growth, regeneration or repair is desired comprising or consisting essentially of administering a therapeutically effective amount of a short chain polyphosphate to the site.

The invention provides a method of treating a subject with a cartilage disease or condition comprising administering to the subject a therapeutically effective amount of a short chain polyphosphate. The invention also provides a method of treating a subject suffering from a cartilage defect comprising administering to the subject a therapeutically effective amount of a short chain polyphosphate. The invention also provides a method for tissue repair utilizing a short chain polyphosphate to repair a cartilage defect or meniscal tear in a joint, such as the knee, comprising administering to the subject a therapeutically effective amount of a short chain polyphosphate. The invention further provides a method for repairing damaged cartilage tissue in a subject comprising administering to the subject a therapeutically effective amount of a short chain polyphosphate.

The invention provides a method of arresting the progress of cartilage disease, or restoring a cartilage that has undergone deformation and/or detrition due to illness or trauma which comprises or consists essentially of administration of a short chain polyphosphate as an active ingredient.

The invention provides a method for regenerative treatment of cartilage disease in a patient, which comprises or consists essentially of administration of a short chain polyphosphate as an active ingredient. The method may involve local administration of a formulation for gradually releasing the short chain polyphosphate at the affected region. The formulation may comprise a biocompatible and biodegradable polymer or macromolecule. In some aspects the method may involve oral administration of a short chain polyphosphate.

The methods of the invention have particular application in conditions such as osteoarthritis. Therefore, the invention provides a method of treating osteoarthritis in a patient comprising or consisting essentially of administering to the subject at least one short chain polyphosphate. In an aspect, the invention provides a method of preserving and stimulating cartilage in a human having osteoarthritis comprising or consisting essentially of administering to said human at least one short chain polyphosphate.

In an aspect, the invention provides a method of restoring or increasing cartilage matrix accumulation or synthesis in a subject having osteoarthritis comprising or consisting essentially of administering to said subject at least one short chain polyphosphate. In an aspect, the invention provides a method of treating osteoarthritis in a subject comprising administering to the subject a therapeutically effective amount of at least one short chain polyphosphate to restore or increase cartilage matrix accumulation or synthesis. In an aspect, the invention provides a method of increasing collagen and proteoglycan content or synthesis in a subject having osteoarthritis comprising or consisting essentially of administering to said subject at least one short chain polyphosphate. In an aspect, the invention provides a method of treating osteoarthritis in a subject comprising or consisting essentially of administering to said subject at least one short chain polyphosphate in a therapeutically effect amount to increase collagen and proteoglycan content or synthesis. In an aspect, the invention provides a method of treating osteoarthritis in a subject comprising or consisting essentially of administering to said subject at least one short chain polyphosphate in a therapeutically effective amount to improve or decrease histological grade of degenerative changes as measured by ICRS scale.

The invention provides a method for treatment of osteoarthritis in a subject, which comprises or consists essentially of administering to the subject a therapeutically effective amount of a medicament comprising at least one short chain polyphosphate.

In an aspect, the invention relates to a method for the treatment of knee or hip osteoarthritis affecting mobility comprising or consisting essentially of administration to a subject, in particular a mammal, of a composition comprising a therapeutically effective amount of at least one polyphosphate. In an embodiment, the subject has responded poorly to lifestyle changes and/or analgesics.

In an aspect, the invention relates to a method for the treatment or preventative treatment of osteoarthritis comprising periodic administration to a subject, in particular a mammal, of a composition comprising an amount of a short chain polyphosphate effective for reducing the progression of cartilage destruction. The period of administration may range from a week to one or more months, or to years.

In an aspect, the invention relates to a method for the treatment or preventative treatment of osteoarthritis comprising continuous administration to a subject, in particular a mammal, of a composition comprising an amount of a short chain polyphosphate effective for reducing the progression of cartilage destruction. The period of continuous administration may range from hours, a day(s) or a week to one or more months to years.

In embodiments of the invention, a short chain polyphosphate is administered by direct injection into the closed cavity of the joint (i.e., intra-articular injection), subcutaneous injection, oral administration or other known methods.

The invention in an embodiment provides a method of treating osteoarthritis in a subject or patient in need thereof, comprising administering a pharmaceutically acceptable composition comprising at least one short chain polyphosphate, preferably into an intra-articular space of a joint of a subject, wherein about 4, 8, 10, 12, 16, or 20 weeks after the administration the patient has substantial improvement in the joint function. The substantial improvement may be measured in a variety of different ways including the Western Ontario and McMaster Universities (WOMAC) score or by a visual analog scale (VAS) score.

In an embodiment, the invention provides a method of treating osteoarthritis, which comprises administering to the affected joint, preferably by intra-articular injection, a therapeutically effective amount of at least one short chain polyphosphate, alone or in combination with at least one other osteoarthritis treatment agent. Osteoarthritis treatment agents include, without limitation, pharmaceutically acceptable viscosupplements, steroidal and non-steroidal anti-inflammatory agents, glucosamines, chondroitins, and the like.

The invention relates to a composition for stimulating or enhancing cartilage repair, slowing or inhibiting cartilage degradation, and/or reducing or inhibiting cartilage mineralization comprising a therapeutically effective amount of a short chain polyphosphate, and a pharmaceutically acceptable carrier, excipient or vehicle.

In an aspect, the invention provides a composition for restoring or increasing cartilage matrix accumulation, increasing collagen and proteoglycan content, and/or improving or decreasing histological grade of degenerative changes as measured by ICRS scale comprising a therapeutically effective amount of a short chain polyphosphate, and a pharmaceutically acceptable carrier, excipient or vehicle.

In an embodiment, the present invention comprises a novel composition useful for treating osteoarthritis in accordance with the present invention. The composition of the invention comprises or consists essentially of at least one short chain polyphosphate and optionally at least one osteoarthritis treatment agent. In one preferred embodiment, a composition comprises or consists essentially of at least one short chain polyphosphate and at least one injectable osteoarthritis treatment agent. Compositions of the invention may also contain other materials such as fillers, stabilizers, coatings, colorizing and flavoring agents, preservatives, fragrances, and other additives known in the art.

In an aspect, the invention relates to a composition for the treatment of osteoarthritis comprising or consisting essentially of an amount of a short chain polyphosphate effective for reducing the progression of cartilage destruction and a pharmaceutically acceptable carrier, excipient or vehicle. In embodiments of the invention, the pharmaceutically acceptable carrier, excipient or vehicle is of a type suitable for the formulation of the composition for an intra-articular or subcutaneous injection or oral administration. The amount of short chain polyphosphate present in each dosage form may range from 0.1 ng to 1000 mg (phosphate (Pi) equivalents), 1 ng to 1000 mg (phosphate equivalents) or 1 ng to 500 mg (Pi equivalents) per dosage, in particular 10 ng to 100 mg per dosage. In embodiments of compositions, dosage forms and formulations of the invention, the amount of short chain polyphosphate ranges from about 0.001 to 100 mg (Pi equivalents). In embodiments of compositions, dosage forms and formulations of the invention, the amount of short chain polyphosphate ranges from about 0.01 to 100 mg (Pi equivalents). In embodiments of compositions, dosage forms and formulations of the invention, the amount of short chain polyphosphate ranges from about 0.01 to 50 mg (Pi equivalents), in particular 0.01 to 20 mg (Pi equivalents), more particularly 0.01 to 10 mg (Pi equivalents).

In one embodiment, the invention provides a composition suitable for intra-articular injection wherein the short chain polyphosphate and optionally an osteoarthritis treatment agent are injectable, preferably in a form suitable for intra-articular injection. In one embodiment, the osteoarthritis treatment agent may comprise at least one corticosteroid such as a glucocorticoid. In a particular embodiment, the injectable osteoarthritis treatment agent is methylprednisolone acetate, more particularly 1-25 mg/mL of injectable methylprednisolone acetate. In another embodiment, the osteoarthritis treatment agent may comprise at least one viscosupplement (i.e., a substance that is used to restore and/or increase the cushioning and lubrication of arthritic synovial fluid).

The invention relates to a use of a short chain polyphosphate as a medicament or in the preparation of a medicament for stimulating cartilage growth, regeneration or repair at a site in a subject where cartilage growth, regeneration or repair is desired. In an aspect, the invention relates to use of a short chain polyphosphate as a medicament for treating osteoarthritis.

The invention also provides methods, compositions and kits for stimulating in vitro formation of cartilage tissue, and in particular stimulating extracellular matrix accumulation in in vitro formed cartilage or ex vivo cultured cartilage.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will be better understood with reference to the drawings in which:

FIG. 1: (A) Glycosaminoglycan and collagen contents and (B) DNA content of full thickness cartilage formed in vitro on membrane inserts after 2 weeks in medium supplemented with different concentrations of polyphosphate (average chain length of 45 phosphate units). The polyphosphate concentration was determined based on the weight of the polyphosphate. The glycosaminoglycan and collagen contents data were normalized to DNA content and presented as percent change compared to non-treated control. Each condition was done in triplicate and the experiment was repeated 3 times. The results were pooled and expressed as mean±standard error of the mean. * indicates a significant change compared to non-treated tissues.

FIG. 2: Glycosaminoglycan and collagen contents of full thickness cartilage formed in vitro on membrane inserts in medium supplemented with polyphosphate (1 mM, calculated based on the phosphate content) of different chain lengths for 2 weeks. The polyphosphate concentration was determined based on the weight of the polyphosphate. The data was normalized to DNA content and presented as percent change compared to non-treated control. Each condition was done in triplicate and the experiment was repeated 3 times. The results were pooled and expressed as mean±standard error of the mean. * indicates a significant increase compared to non-treated tissues.

FIG. 3: (A) glycosaminoglycan, (B) collagen and (C) DNA contents of full thickness cartilage formed in vitro on membrane inserts in medium with and without supplementation with 1 mM polyphosphate (average chain length of 45 phosphate units) at 0, 1, 2 and 4 weeks following initiation of the treatment. The polyphosphate concentration was determined based on the weight of the polyphosphate. The matrix content data was normalized to DNA content. Each condition was done in triplicate and the experiment was repeated 3 times. The results are from one representative experiment and expressed as mean±standard deviation. * indicates a significant change compared to non-treated tissues.

FIG. 4: (A) Glycosaminoglycan and collagen contents of full thickness cartilage formed in vitro on membrane inserts for 4 weeks in medium supplemented with 1 mM polyphosphate (average chain length of 45 phosphate units) for various time periods up to 4 weeks. The polyphosphate concentration was determined based on the weight of the polyphosphate. The data was normalized to DNA content. Each condition was done in triplicate and the experiment was repeated 3 times. The results were pooled and expressed as mean±standard error of the mean. * indicates a significant increase compared to non-treated tissues. (B-E) Histological appearance of cartilage formed after 4 weeks of culture from full thickness chondrocytes on membrane inserts in medium not supplemented (B) or supplemented for the first week (C), the first 2 weeks (D) or the entire 4 weeks (E) with 1 mM polyphosphate and stained with DAPI. Tissues were visualized by epifluorescence microscopy.

FIG. 5: DNA, glycosaminoglycan and collagen contents of cartilage formed by full thickness (FT), superficial-mid zone (SMZ) or deep zone (DZ) chondrocytes cultured in vitro on membrane inserts in medium with and without supplementation with 1 mM polyphosphate (average chain length of 45 phosphate units) at 2 weeks. The polyphosphate concentration was determined based on the weight of the polyphosphate. The matrix content data was normalized to DNA content. All data from cultures treated with polyphosphate is expressed as a percentage of untreated cultures for each chondrocyte subpopulation. Each condition was done in triplicate and the experiment was repeated 3 times. The results are from one representative experiment and expressed as mean±standard deviation.

FIG. 6: (A) Glycosaminoglycan and collagen contents and (B) DNA content of native articular cartilage cultured ex vivo on membrane inserts for 1 week in medium supplemented with 1 mM or 2 mM polyphosphate (average chain length of 45 phosphate units). The polyphosphate concentration was determined based on the weight of the polyphosphate. The data was normalized to DNA content and presented as percent change compared to non-treated control. Each condition was done in triplicate and the experiment was repeated 3 times. The results were pooled and expressed as mean±standard error of the mean. * indicates a significant increase compared to non-treated tissues. (C-E) Histological appearance of native articular cartilage cultured ex vivo for 1 week in medium not supplemented (C) or supplemented with 1 mM (D) or 2 mM (E) polyphosphate and stained with toluidine blue. Tissues were visualized by light microscopy. Arrow indicates new tissue.

FIG. 7 shows images of the histological appearance of polyphosphate treated and control guinea pig joints. Tibial plateaux were harvested 2 months after medial menisectomy, decalcified and processed for histological evaluation. Photomicrographs of control (A,B) and polyphosphate treated (C,D) joint surfaces showing mild osteoarthritic changes in the polyphosphate treated joint and severe osteoarthritic changes with loss of cartilage in the untreated control (PBS). Arrow indicates site of osteoarthritic change. (toluidine blue stain, original magnification ×12.5 (A,C) and ×50 (B,D).

FIG. 8 is a graph illustrating the average scores of the tibial plateau histological features in polyphosphate treated (4 mM) and control (PBS) guinea pigs. * indicates a significant difference, p<0.05, n=9

FIG. 9 is a graph illustrating the GAG/DNA content of articular cartilage from the medial femoral condyle of polyphosphate treated and control guinea pigs two months after menisectomy. * indicates a significant difference, p<0.05, n=10

FIG. 10 is a graph illustrating Collagen/DNA content of articular cartilage from the medial femoral condyle of polyphosphate treated and control guinea pigs two months after menisectomy. * indicates a significant difference, p<0.05, n=10

FIG. 11 is a graph illustrating glycosaminoglycan content of chondrocytes cultured on membrane inserts for 10 days in DMEM+20% FBS supplemented with 0.5 mM inorganic polyphosphate of different chain lengths. The data was normalized to DNA content μg glycosaminoglycan per μg of DNA. Each condition was done in triplicate. The results are expressed as mean±standard deviation.

### DETAILED DESCRIPTION OF THE INVENTION

### Glossary

“Short chain polyphosphates” include polymers comprising 3 or more phosphate (Pi) units, 5 or more Pi units, 3 to 100 Pi units, 5 to 100 Pi units, 5 to 85 Pi units, or 5 to 75 phosphate (Pi) units, in particular 40 to 50 Pi units, linked together by phosphoanhydride bonds. In embodiments, the short chain polyphosphate is an inorganic polyphosphate of at least 5 Pi units. In embodiments, the short chain polyphosphate is an inorganic polyphosphate of greater than 5 Pi units. In embodiments, the short chain polyphosphate is an inorganic polyphosphate of at least 6, 10, 15, 20, 25, 30, 50 or 100 Pi units. In embodiments, the short chain polyphosphate is an inorganic polyphosphate of average chain lengths ranging from 5 to 200 Pi units, 5 to 150 Pi units, 5 to 100 Pi units, 5 to 90 Pi units, 5 to 85 Pi units, 5 to 75 phosphate units or 5 to 60 Pi units. In embodiments, the short chain polyphosphate is an inorganic polyphosphate of average chain lengths ranging from 10 to 200 Pi units, 10 to 150 Pi units, 10 to 100 Pi units, 10 to 90 Pi units, 10 to 85 Pi units, 10 to 75 phosphate units or 10 to 60 Pi units. In embodiments, the short chain polyphosphate is an inorganic polyphosphate of average chain lengths ranging from 15 to 200 Pi units, 15 to 150 Pi units, 15 to 100 Pi units, 15 to 90 Pi units, 15 to 85 Pi units, 15 to 75 phosphate units or 15 to 60 Pi units. In embodiments, the short chain polyphosphate is an inorganic polyphosphate of average chain lengths ranging from 20 to 200 Pi units, 20 to 150 Pi units, 20 to 100 Pi units, 20 to 90 Pi units, 20 to 85 Pi units, 20 to 75 phosphate units or 20 to 60 Pi units. In embodiments, the short chain polyphosphate is an inorganic polyphosphate of average chain lengths ranging from 30 to 90 Pi units, 30 to 60 phosphate units, more particularly 40 to 50 phosphate units. In particular embodiments, the short chain polyphosphate comprises an average of about 45 Pi units. In some embodiments, the term may also include modified forms of these polymers, in particular modified forms that have reduced resistance to phosphatases.

The term “short chain polyphosphate” includes complexes comprising the polyphosphate polymers (e.g. 5 or more Pi units linked together by phosphoanhydride bonds) complexed with alkali earth metals, alkaline earth metals or transition metals. In aspects of the invention, the complexes comprise cations such as Fe³⁺, Fe²⁺, Pb²⁺, Co²⁺, cu²⁺, UO²⁺, Ni²⁺, Zn²⁺, Mn²⁺, Mg²⁺, Ca²⁺, Sr²⁺, Ba²⁺ and Ag²⁺. In a particular aspect of the invention, the short chain polyphosphate is a calcium polyphosphate.

Short chain polyphosphates can be produced using biological and synthetic methods. For example, short chain polyphosphates may be isolated from cells of living organisms, including without limitation, bacteria and yeast, in particular E. coli and S. cerevisiae. Short chain polyphosphates present in cells may be isolated by extraction using basic solutions (e.g. sodium hydroxide, sodium chloride, sodium carbonate, sodium hypochlorite or potassium hydroxide) and/or acidic solutions (e.g. trichloroacetic acid, perchloric acid), hot water, or cold distilled water following treatment with alcohol and ether (see for example, Kulaev, I S, Vagabov, V M and Kulakovskaya T V; The Biochemistry of Inorganic Polyphosphate, Second Edition, 2004, John Wiley & Sons, Ltd, Chichester, England; Lorenz, B, Schroder, H C, Muller W E G, Methods of Investigation of Inorganic Polyphosphates. In Inorganic Polyphosphates: Biochemistry, Biology, Biotechnology; Schroder, H C, Muller, W E G, Eds: Progress in Molecular and Subcellular Biology, Vol. 23; Springer-Verlag, Heidelberg, Germany, 1999; and Omelon, S J and Grynpas, M D, Chem. Rev. 2008, 108, 4694-4715). Short chain polyphosphates can also be produced by dehydration at high temperatures or they can be enzymatically synthesized, for example, using select kinases (see, Griffith, E J, Phosphate Fibers; Plenum Press; New York, 1995; The Biochemistry of Inorganic Polyphosphate, Second Edition, Eds: Kulaev, I S, Vagabov, V M, and Kulakovskaya, T V; 2004, John Wiley & Sons, Ltd, Chichester, England; Muhlradt, P F, Journal of General Microbiology, 1971, 68:115-122). Complexes of polyphosphate polymers with metal ions may be prepared using methods known in the art (Van Wazer J R, and Campanella D A, J. Am. Chem Soc, 1950, 72:655)

In an aspect of the invention, the short chain polyphosphate is a calcium polyphosphate produced by calcination of calcium phosphate monobasic monohydrate (Pilliar, R M et al, 2001, Biomaterials, 22:963-972).

The composition of the short chain polyphosphates may be confirmed, for example by ³¹P NMR, mass spectrometry, chromatography, hydrolytic degradation assays, colorimetry, solubility fractionation, enzyme assays, gel electrophoresis, infrared, cytochemical methods, thermal analysis and X-ray diffraction (see review by Omelon, S J and Grynpas, M D, Chem. Rev. 2008, 108, 4694-4715, and Example 4).

A “therapeutically effective amount” of a short chain polyphosphate may be administered to a subject. This quantity provides greater cartilage growth, repair or regeneration in the presence of the short chain polyphosphate than in its absence. This quantity may additionally or alternatively result in reduction or alleviation of the pain and/or lack of function associated with the cartilage damage. A short chain polyphosphate is generally administered for a sufficient period of time to achieve a desired therapeutic effect. The amount of compound administered will depend on a number of factors including without limitation, the amount of cartilage growth that is desired, the health, size, weight, age and sex of the subject and the release characteristics of the pharmaceutical formulation. A therapeutically effective amount may be administered intermittently or continuously, and it may be administered by any means which produce a therapeutic effect, including without limitation, orally, intranasally, by inhalation, intraperitoneally, subcutaneously, intramuscularly, transdermally, sublingually, intravenously or intra-articularly. In particular embodiments of the invention, a therapeutically effective amount is administered by direct application to the site in need of cartilage growth, regeneration or repair.

Generally, a therapeutically effective amount is between about 0.1 ng and about 1000 mg or about 1 ng and about 1000 mg, in particular between about 1 ng and about 100 mg, about 5 ng and about 100 mg or about 10 ng and about 100 mg. In aspects of the invention, a therapeutically effective amount of polyphosphate in a dosage form ranges from about 0.1 ng to about 1000 mg or about 1 ng to about 1000 mg per dosage, in particular about 5 ng to about 100 mg, 10 ng to 100 mg, 10 ng to 50 mg, 10 ng to 25 mg 10 ng to 10 mg or 10 ng to 5 mg per dosage.

In embodiments of compositions, dosage forms and formulations of the invention, the amount of polyphosphate ranges from about 0.001 to 100 mg (Pi equivalents). In embodiments of compositions, dosage forms and formulations of the invention, the amount of polyphosphate ranges from about 0.01 to 100 mg (Pi equivalents). In embodiments of compositions, dosage forms and formulations of the invention, the amount of polyphosphate ranges from about 0.1 to 100 mg (Pi equivalents).

In embodiments of compositions, dosage forms and formulations of the invention, the amount of polyphosphate ranges from about 0.001 to 50 mg (Pi equivalents), in particular 0.001 to 20 mg (Pi equivalents), more particularly 0.001 to 10 mg (Pi equivalents). In embodiments of compositions, dosage forms and formulations of the invention, the amount of polyphosphate ranges from about 0.01 to 50 mg (Pi equivalents), in particular 0.01 to 20 mg (N equivalents), more particularly 0.01 to 10 mg (Pi equivalents). In embodiments of compositions, dosage forms and formulations of the invention, the amount of polyphosphate ranges from about 0.05 to about 5 mg (Pi equivalents). In embodiments of compositions, dosage forms and formulations of the invention, the amount of polyphosphate ranges from about 0.05 to about 2 mg (Pi equivalents). In embodiments of compositions, dosage forms and formulations of the invention, the amount of polyphosphate ranges from about 0.03 to 10 mg (Pi equivalents). In embodiments of compositions, dosage forms and formulations of the invention, the amount of polyphosphate ranges from about 0.03 to 5 mg (Pi equivalents).

The concentration of polyphosphate based on polyphosphate content (using for example an enzymatic method such as in Example 4) in a dosage form may be at least about 0.01 mM, in particular at least about 0.05 mM. In embodiments of compositions, dosage forms and formulations of the invention, the amount of polyphosphate ranges from about 0.01 mM to about 50 mM, in particular about 0.01 mM to about 20 mM, more particularly about 0.01 mM to about 10 mM. In embodiments of compositions, dosage forms and formulations of the invention, the amount of polyphosphate ranges from about 0.05 mM to about 10 mM, in particular about 0.05 mM to about 5 mM, more particularly about 0.05 mM to about 2 mM. In embodiments of compositions, dosage forms and formulations of the invention, the amount of polyphosphate ranges from about 0.1 mM to about 10 mM, in particular about 0.1 mM to about 5 mM, more particularly about 0.1 mM to about 2 mM.

The terms “administering” and “administration” refer to the process by which a therapeutically effective amount of a compound or composition contemplated herein is delivered to a subject for prevention and/or treatment purposes. Compositions are administered in accordance with good medical practices taking into account the subject's clinical condition, the site and method of administration, dosage, patient age, sex, body weight, and other factors known to physicians.

The term “treating” refers to reversing, alleviating, or inhibiting the progress of a disease, or one or more symptoms of such disease, to which such term applies. Depending on the condition of the subject, the term also refers to preventing a disease, and includes preventing the onset of a disease, or preventing the symptoms associated with a disease. The treatment may either be performed in an acute or chronic way. The term also refers to reducing the severity of a disease or symptoms associated with such disease prior to affliction with the disease. Such prevention or reduction of the severity of a disease prior to affliction refers to administration of a short chain polyphosphate or composition of the present invention to a subject that is not at the time of administration afflicted with the disease. “Preventing” also refers to preventing the recurrence of a disease or of one or more symptoms associated with such disease. The terms “treatment” and “therapeutically,” refer to the act of treating, as “treating” is defined above.

In aspects of the invention treatment includes protection of cartilage from wear, inhibition of degenerative changes in cartilage as a result of disease, trauma or inflammation, repair of cartilage injury lesions and inhibition of inflammation and pain.

In aspects of the invention, treatment comprises regeneration of cartilage tissue, including restoring the function of a cartilage injury lesion for which cartilage function has been impaired or lost. Restoration of cartilage function does not require that function be completely restored, rather that function be restored to a greater degree than the state of the lesion prior to application of the methods and compositions of the present invention.

The terms “subject”, “individual”, or “patient” are used interchangeably herein and refer to an animal including a warm-blooded animal such as a mammal, which is afflicted with or suspected of having or being pre-disposed to a disease disclosed herein. Mammal includes without limitation any members of the Mammalia. In aspects of the invention, the terms refer to a human. The terms also include domestic animals bred for food or as pets, including horses, cows, sheep, poultry, pigs, cats, dogs, and zoo animals, goats and apes (e.g. gorilla or chimpanzee). Typical subjects for treatment include persons susceptible to, suffering from or that have suffered a disease or condition requiring repair or regeneration of cartilage, inhibition or reduction of cartilage mineralization, and/or restored or increased cartilage matrix accumulation. A cartilage disease includes a disease that occurs due to injury of cartilage, cartilage tissue and/or joint tissue by mechanical means or inflammation. In aspects of the invention the injury is in a joint (e.g., in the knee, shoulder, ankle, elbow, wrist, fingers and the like).

In embodiments of the invention the subjects are suspectible to, or suffer from osteoarthritis or have traumatized their cartilage. In embodiments of the invention the osteoarthritis occurs in joints, including without limitation those of the knees, shoulders, hips, lower back, ankles, wrists, fingers and the like. In embodiments of the invention the subject suffers from osteoarthritis of the hip or knee. In embodiments of the invention the subject suffers from osteoarthritis affecting hip or knee mobility. In embodiments of the invention, the subject requires arresting of the progress of a cartilage disease or restoration of cartilage that has undergone deformation and/or detrition due to illness or trauma. In embodiments of the invention the subject suffers from an inflammatory arthritis, in particular rheumatoid arthritis.

In embodiments of the invention the subject suffers from rheumatoid arthritis, osteoarthritis, psoriatic arthritis, reactive arthritis, systemic sclerosis, systemic lupus erythematosus or relapsing polychondritis.

The term “pharmaceutically acceptable carrier(s), excipient(s), or vehicle(s)” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. Examples of carriers etc. include but are not limited to saline, buffered saline, dextrose, water, glycerol, ethanol, hyaluronate and combinations thereof. The use of such media and agents for an active substance is well known in the art. In some embodiments of the invention, a carrier may be a solid support including beads, discs, spheres, substrates, and the like. Suitable pharmaceutical carriers, excipients, and vehicles are described in the standard text, Remington: The Science and Practice of Pharmacy, 21^{st} Edition. University of the Sciences in Philadelphia (Editor), Mack Publishing Company.

### Methods and Compositions

The invention provides methods for stimulating cartilage growth, repair or regeneration by administering a short chain polyphosphate. The invention also provides methods for stimulating cartilage growth, repair or regeneration by administering a short chain polyphosphate comprising at least or more than 5, 6, 10, 15, 20, 50 or 100 Pi units.

A short chain polyphosphate or composition of the present invention may be administered by any means that produce contact of the active agent(s) with the agent's sites of action in the body of a subject or patient. A short chain polyphosphate or composition of the present invention may be administered by conventional methods including without limitation orally, intranasally, by inhalation, intraperitoneally, subcutaneously, intramuscularly, transdermally, sublingually, intravenously or intra-articularly.

A short chain polyphosphate may be administered to a subject as one component in a pharmaceutical composition to a site in need of treatment, including sites in need of cartilage growth, repair or regeneration. The treatment site may be, for example, in a joint (e.g., in the knee, shoulder, ankle, elbow, wrist, fingers and the like). The compound is administered in sufficient proximity to the site in need of treatment so that cartilage growth or cartilage regeneration occurs at the site (e.g., there is a greater amount of cartilage growth or better quality of cartilage growth in the presence of the short chain polyphosphate than in its absence).

A pharmaceutical composition of the invention may comprise a short chain polyphosphate and a pharmaceutically acceptable carrier, excipient or vehicle.

In an aspect, a pharmaceutical composition of the present invention is a solution comprising a short chain polyphosphate and a suitable carrier, which is applied directly to or in proximity to the treatment site. A solution can be administered using conventional methods such as intra-articularly by syringe, by syringe in close proximity to the damaged tissue, or through a surgical opening. Examples of carriers that may be used for solution compositions include without limitation, physiological saline, bacteriostatic saline, phosphate-buffered saline, Hank's solution, Ringer's-lactate and the like.

In other aspects, a pharmaceutical composition of the invention is an oral dosage form such as a tablet, capsule (each of which includes sustained release or timed release formulations), pill, powder, granule, elixir, tincture, suspension, syrup, or emulsion. In an embodiment, the invention provides an oral dosage form, in particular a tablet, comprising therapeutically effective amounts of a short chain polyphosphate and optionally an osteoarthritis treatment agent.

The invention contemplates sustained release formulations comprising a short chain polyphosphate. A “sustained release formulation(s)” refers to a dosage form that releases the active compound (i.e. short chain polyphosphate) for many hours, or days to months. In an aspect, a sustained release formulation includes a component for slowing disintegration or dissolution/degradation of the short chain polyphosphate. In aspects of the invention, a sustained release formulation may be engineered with or without an initial delay period. A sustained release formulation may exhibit Tₘₐₓ values of at least two, four, six, or eight hours or more and in particular up to about 24 hours or more, for once daily (qd) or twice per day (bid) dosing. These formulations may continuously release the short chain polyphosphates for sustained periods of at least about 4 to 6 hours or more, preferably about 8 hours or more and, in particular embodiments, about 12 hours or more, about 12 hours to 24 hours, about 20 hours to 24 hours, about 24 to 48 hours, about 36 to 72 hours or greater.

A sustained release formulation can be in a variety of physical structures or forms, including without limitation, suspensions, solutions, tablets, or gels. A sustained release formulation is preferably selected that results in administration of a minimum number of doses. In aspects of the invention, a sustained release formulation may be administered as one dose about every 2 months, 4 months, 6 months or 12 months. In an aspect, a sustained release formulation comprises a biocompatible and biodegradable polymer, in particular a water-soluble polymer, such as polylactic acid, lactic acid-glycolic acid copolymer and 2-hydroxybutyric acid-glycolic acid copolymer. In aspects of the invention, the sustained release formulation is a microsphere preparation.

In another aspect, the pharmaceutical composition comprises a short chain polyphosphate and an implantable biocompatible carrier. A biocompatible carrier is preferably selected that is non-toxic, non-inflammatory, and non-immunogenic and provides for sustained release of the polyphosphate at the treatment site. Examples of biocompatible carriers include without limitation, synthetic biodegradable polymers such as poly α-hydroxy esters (in particular, polylactic acid/polyglycolic acid homo and copolymers, and polyanhydrides). Polylactic acid/polyglycolic acid (PLGA) homo and copolymers are well known in the art as sustained release vehicles (see for example, U.S. Pat. Nos. 6,013,853, 5, 607,474 and 5,876,452. A poly(ethylene glycol) may be added to the polymer blend to attenuate the release profile of a polyphosphate [see for example, Cleek et al., J. Control Release 48:259 (1997)]. Hydrophobic or hydrophilic monomers such as sebacic acid and 1,3-bis(p-carboxyphenoxy)propane may also be included in compositions to control degradation and release properties of polyanhydrides [see, for example, Leong et al., J. Biomed. Mater. Res. 19:941 (1985)]. To improve mechanical strength, polyanhydrides can be copolymerized with imides.

In another aspect, a pharmaceutical composition of the invention comprises a short chain polyphosphate and a carrier which is a viscous solution or gel suitable for intra-articular injection or injection at the site in need of treatment without the need for invasive surgery. Suitable viscous solution or gel carriers include hyaluronic acid [e.g., ORTHOVISC® (Anika), SYNVISC® (Biomatrix), HYALGAN® (Fidia), ARTZ® (Seikagaku), and DUROLANE® (Smith & Nephew)] and pluronic gel.

Moreover the present invention provides a composition comprising or consisting essentially of a short chain polyphosphate for use as a nutraceutical or for the manufacture of a nutraceutical for the improvement of health or for stimulating cartilage repair, reducing and/or stimulating cartilage growth, regeneration or repair in a subject, and treating diseases involving cartilage defects as well as diseases including osteoarthritis. A composition of the invention may be in the form of a dietary supplement, or as a food, feed or pet food ingredient. A dietary supplement may also include without limitation, vitamins, minerals, herbs or other botanicals, amino acids, substances such as enzymes, organ tissues, glandulars, and metabolites, and substances to prevent digestion or degradation. A dietary supplement may be in any form including without limitation tablets, capsules, softgels, gelcaps, liquids, bars or powders. The label of a dietary supplement in general does not represent the product as a conventional food or an individual item of a meal or diet. A composition of the invention can also be used for the manufacture of a functional food product to prevent conditions or diseases involving cartilage defects as well as diseases including osteoarthritis, or for health maintenance in subjects with conditions or diseases involving cartilage defects as well as diseases including osteoarthritis. A composition of the invention can be used in a functional food product capable of providing a health benefit to the consumer, in particular the health benefit may be selected from the prevention of conditions or diseases involving cartilage defects as well as diseases including osteoarthritis, or for health maintenance in subjects with conditions or diseases involving cartilage defects as well as diseases including osteoarthritis. The invention also provides a process to prepare a food product, beverage product or dietary supplement comprising production of a composition of the invention comprising a short chain polyphosphate and incorporation of said composition into a food product, beverage product or dietary supplement.

The invention contemplates methods for stimulating cartilage repair, reducing and/or stimulating cartilage growth, regeneration or repair in a subject, and treating diseases involving cartilage defects as well as diseases including osteoarthritis.

In an aspect, the invention relates to a method for repairing a cartilage defect, preferably an articular cartilage defect, at a pre-determined site in a mammal (preferably humans) comprising administering a short chain polyphosphate. A cartilage defect can be identified during arthroscopic examination or during open surgery of the joint. Defects can also be identified using computer aided tomography (CT scanning), X-ray examination, magnetic resonance imaging (MRI), analysis of synovial fluid or serum markers, or other procedures known in the art. Treatment of defects can be carried out during an arthroscopic or open joint procedure. Once a defect is identified it may be treated using a method of the invention.

Joint pain, function and stiffness may be assessed prior to, during and after a treatment of the present invention using the visual analog scale (VAS) for pain, and the Western Ontario and McMaster Universities (WOMAC) osteoarthritis index, which assesses pain, function and stiffness in arthritic joints. (See “Clinical Development Programs for Drugs, Devices, and Biological Products Intended for the Treatment of Osteoarthritis, U.S. Dept. of Health and Human Services, Food and Drug Administration, July 1999” describing clinical endpoints.)

The methods and compositions described herein can be used in the treatment of both partial- and full-thickness defects of articular cartilage. These defects can arise during trauma of the joint or during the late stages of degenerative joint diseases (e.g. osteoarthritis). Partial-thickness defects are restricted to the cartilage tissue itself and include fissures, clefts, or erosions whereas full-thickness defects involve the full thickness of the cartilage. These defects are usually caused by disease, trauma or mechanical derangements of the joint which in turn induce wearing of the cartilage tissue within the joint.

Arthritic sites in need of cartilage growth, repair or regeneration in subjects with osteoarthritis may be treated using a method or composition of the invention. The regeneration of damaged cartilage and the growth of new cartilage at these arthritic sites in a subject may relieve the pain and restore the loss of function associated with osteoarthritis. Trauma from injury (e.g. sports injuries), disease or surgery may also provide sites of cartilage damage which can be repaired or regenerated using methods and compositions of the invention.

The methods and compositions of the invention may be particularly useful in the treatment of knee or hip osteoarthritis, and in particular in subjects that have responded poorly to lifestyle changes and/or analgesics, and where surgery may be the only option.

The methods and compositions of the invention may be used in combination with known treatments for replacement of cartilage tissue. The methods and compositions disclosed herein may be used in combination with marrow-stimulation techniques such as microfracture, abrasion and drilling which results in fibrocartilage repair tissue. They may also be used in combination with osteochondral transfer/mosaicplasty which involves transplanting osteochondral plugs obtained from donor sites within the same joint into a defect site. Further, the methods and compositions of the invention may be used in combination with autologous chondrocyte implantation which involves using an individual's own cartilage to provide the cells that are placed into the cartilage defect under a flap, which can be either periosteum or a collagen membrane. Still further, the methods and compositions of the invention may be used in combination with tissue engineered cartilage, and short chain polyphosphates may be used in methods for preparing engineered cartilage [see, for example, U.S. Pat. Nos. 6,464,729, 5,326,357, US Published Application Nos. 20070071733 and 20070071733, and the Example herein].

In aspects of the invention for treating osteoarthritis, the methods and compositions of the invention may be used in combination with osteoarthritis treatment agents. Examples of osteoarthritis treatment agents include, without limitation, pharmaceutically acceptable viscosupplements, non-steroidal anti-inflammatory drugs (NSAIDS) such as ibuprofen, naproxen, and COX-2 inhibitors; stem cell therapies; analgesics such as aspirin and acetaminophen; glycans, including glucosamines, e.g. glucosamine sulfate and glucosamine hydrochloride; and proteoglycans, such as chondroitin compounds, as well as various other known narcotics, steroids, antibiotics, immunomodulators, penicillamine, and the like. In embodiments of the invention, the osteoarthritis treatment agent is a viscosupplement, including without limitation hylan, hyaluronic acid and other hyaluronan (sodium hyaluronate) compounds, which are natural complex sugars of the glycosaminoglycan family. In particular embodiments, the viscosupplement is a commercially available hyaluronan viscosupplement such as Synvisc® (Genzyme, Cambridge, Mass.), Hyalgan ® (Fidia Pharma USA Inc., Parsippany, N.J.). Supartz® (Smith & Nephew Orthopaedics, London, UK), Durolane® (Smith & Nephew Orthopaedics, London, UK), and Orthovisc® (DePuy Mitek, Inc., Raynham, Mass.).

In aspects of the invention, the osteoarthritis treatment agent is a biologic agent such as recombinant human fibroblast growth factor 18 (rhFGF-18).

In aspects of the invention, the osteoarthritis treatment agent is Chondrogen® (Osiris Therapeutics, Columbia, Md.) which is an intra-articular injectable formulation comprising adult mesenchymal stem cells.

In aspects of the invention, the osteoarthritis treatment agent is a hyaluronan viscosupplement, in particular Synvisc® (Genzyme, Cambridge, Mass.).

In some aspects of the invention, the short chain polyphosphate, methods, dosage forms and compositions of the invention are used in combination with surgical treatments including without limitation debridement and lavage, alignment, marrow stimulation techniques, osteochondral autograft and autogenous chondrocyte implantation (see for example, Articular Cartilage Lesions, A Practical Guide to Assessment and Treatment, Eds: B J Cole and M M Malek, 2004 Springer-Verlag, New York).

In some aspects of the invention, a short chain polyphosphate or pharmaceutical composition of the invention may also be incorporated in a scaffold or matrix for release or implant into a cartilage defect.

### In Vitro and Ex Vivo Methods, Compositions and Kits

The invention also provides methods for stimulating in vitro formation of cartilage tissue, and in particular stimulating extracellular matrix accumulation in in vitro formed cartilage or ex vivo cultured cartilage.

In an aspect, the invention provides a method of stimulating production of cartilage extracellular matrix components in bioengineered cartilage or ex vivo cultured cartilage comprising culturing the cartilage in the presence of a short chain polyphosphate.

In an aspect, the invention provides a method of promoting glycosaminoglycans (GAG) and collagen accumulation in the extracellular matrix of in vitro formed cartilage or ex vivo cultured cartilage comprising culturing the cartilage in the presence of a short chain polyphosphate.

In an aspect, the invention provides a method of stimulating, enhancing or improving matrix accumulation of in vitro formed cartilage or ex vivo cultured cartilage comprising culturing the cartilage in the presence of a short chain polyphosphate.

In an aspect, the invention provides a method of enhancing or improving thickness of in vitro or ex vivo cultured cartilage comprising culturing the cartilage in the presence of a short chain polyphosphate.

In aspects of the invention, a process is provided for culturing cartilage tissue in vitro or culturing native cartilage ex vivo characterized by exogenously administering a short chain polyphosphate.

In an aspect of the invention, a method is provided for enhancing or improving matrix accumulation of a bioengineered cartilage comprising: culturing chondrocytes on a substrate in vitro in the presence of an amount of a short chain polyphosphate effective to produce a bioengineered cartilage tissue with an increased thickness or extracellular matrix compared to bioengineered cartilage tissue cultured in the absence of the short chain polyphosphate. In embodiments, the dry weight, proteoglycan content, and/or collagen when corrected for cellularity of the bioengineered cartilage are increased compared to bioengineered cartilage grown in the absence of the short chain polyphosphate.

Suitable substrates include bone, engineered biomaterials and porous tissue culture inserts, for example filter inserts. The substrate is optionally coated with an attachment factor. In an embodiment, the chondrocytes are plated on a substrate preferably a porous tissue culture insert, for example a MILLICELL®-CM insert, which has been coated with an attachment factor. [See for example, U.S. Pat. Nos. 6,464,729 and 5,326,357 for detailed methods for culturing cartilage tissue in vitro.]

Different chondrocyte subpopulations may be used in a method of the invention to produce a bioengineered cartilage. For example, the chondrocytes may be from the deep zone or superficial-mid zone of articular cartilage. In embodiments of the invention, the chondrocytes are deep zone chondrocytes.

The invention also provides a method of stimulating matrix accumulation in native cartilage comprising culturing excised native cartilage ex vivo in the presence of a short chain polyphosphate. In an aspect, the method results in matrix deposition primarily on the deep-zone face of the cartilage.

The short chain polyphosphate may be administered at any phase but generally it is administered during the in vitro maturation phase, which may be 3, 4, 5, or 6 days after initiation of the culture.

In the methods of the invention, the short chain polyphosphate(s) may be applied continuously or periodically. A period of continuous administration may range from day(s) to week(s), for example 1-5 weeks and in particular 3-4 weeks.

In embodiments of the in vitro and ex vivo methods of the invention, the short chain polyphosphate has an average chain length of at least 5 phosphate units, at least 10 phosphate units, between 5 and 100 phosphate units, between 5 and 85 phosphate units, between 30 to 50 phosphate units, in particular 40 to 50 phosphate units, more particularly 45 phosphate units.

In embodiments of the in vitro and ex vivo methods of the invention, the short chain polyphosphate is an inorganic polyphosphate of greater than 5 Pi units. In embodiments of the invention, the short chain polyphosphate is an inorganic phosphate of at least 6, 10, 15, 20, 25, 30, 50 or 100 Pi units. In embodiments, the short chain polyphosphate is an inorganic polyphosphate of average chain lengths ranging from 10 to 200 Pi units, 10 to 150 Pi units, 10 to 100 Pi units, 10 to 90 Pi units, 10 to 85 Pi units, 10 to 75 phosphate units or 10 to 60 Pi units. In embodiments, the short chain polyphosphate is an inorganic polyphosphate of average chain lengths ranging from 20 to 200 Pi units, 20 to 150 Pi units, 20 to 100 Pi units, 20 to 90 Pi units, 20 to 85 Pi units, 20 to 75 phosphate units or 20 to 60 Pi units. In embodiments, the short chain polyphosphate is an inorganic polyphosphate of average chain lengths ranging from 30 to 90 Pi units, 30 to 60 phosphate units, more particularly 40 to 50 phosphate units.

In embodiments, the amount administered ranges from 0.001 mM to 100 mM, 0.001 mM to 50 mM, 0.001 mM to 20 mM, 0.05 mM to 100 mM, 0.05 mM to 50 mM 0.05 mM to 10 mM, or 0.1 mM to 10 mM of short chain polyphosphate amount.

The invention also provides a composition comprising an amount of at least one short chain polyphosphate effective for stimulating in vitro formation of cartilage tissue. In an aspect the composition comprises an amount of short chain polyphosphate effective to stimulate extracellular matrix accumulation in in vitro formed cartilage or ex vivo cultured cartilage.

The invention also provides a kit for stimulating in vitro formation of cartilage tissue comprising at least one short chain polyphosphate. In an embodiment, the kit comprises reagents for methods of the present invention for stimulating in vitro formation of cartilage tissue. A kit may also include instructions for use in these methods.

The following non-limiting examples illustrate the present invention:

### Example 1

### Inorganic Polyphosphate Stimulates Cartilage Matrix Deposition

Applicants have observed that cartilage tissues treated with condensed phosphate have an increased amount of tissue compared to untreated controls. It is hypothesized that the exogenous administration of inorganic polyphosphate to hyaline-like cartilage forming in vitro will have anabolic effects on the chondrocytes resulting in increased extracellular matrix accumulation. In this study, the effect of inorganic polyphosphates of average chain lengths ranging from 5 to 75 phosphate units on matrix accumulation within in vitro-formed cartilage was examined along with the ability of chondrocytes to metabolise condensed phosphate through enzymes with exopolyphosphatase activity. The responsiveness of different articular chondrocytes zonal subpopulations cultured in vitro and of native cartilage samples cultured ex vivo to polyphosphate was also investigated. Identifying signaling molecules that positively impact the matrix composition of articular cartilage and can be metabolised by chondrocytes to re-establish homeostasis is essential to the successful engineering of cartilage for joint repair.

The following materials and methods were used in the study described in this Example.

### Substrates:

Membrane inserts (Millicell-CM®, Millipore Corp., Bedford, Mass., USA) were coated with type II collagen (0.5 mg/ml in 0.1N acetic acid; Sigma Chemical Co., St. Louis, Mo., USA) and dried overnight. The membrane inserts were UV sterilized for 30 minutes and soaked in Ham's F12 for 30 minutes prior to cell culturing.

### Tissue Culture:

Cartilage was aseptically excised from the full thickness of metacarpal-phalangeal articular cartilage from 9 to 12 months old calves within 24 hours of death as previously described [5]. For the experiment investigating the response of chondrocytes zonal subpopulations to inorganic polyphosphate, cartilage was excised as the superficial-mid zone (top 70%) and the deep-zone (bottom 30%). Chondrocytes were harvested from the tissue by sequential enzymatic digestion (0.5% protease (Sigma Chemical Co., St. Louis, Mo., USA) for 2 hours followed by 0.1% collagenase (Roche Diagnostics GmbH, Mannheim, Germany) overnight). The cells were then seeded on top of membrane inserts (1×10⁶ cells per construct) in Ham's F-12 supplemented with 5% fetal bovine serum (FBS) (HyClone, Logan, Utah, USA) and incubated at 37° C. in an atmosphere characterized by 95% relative humidity and 5% CO₂. On day 5, the serum concentration was changed to 20% and supplemented with ascorbic acid (100 μg/ml, Sigma Chemical Co., St. Louis, Mo., USA). At this time, cultures were incubated in the presence or absence of various concentrations of sodium phosphate glasses (inorganic polyphosphate; Sigma Chemical Co., St. Louis, Mo., USA) or sodium phosphate buffer. The concentrations of inorganic polyphosphate utilized for each experiment are calculated based on the phosphate content. Cultures were grown for up to 4 weeks following initiation of the inorganic polyphosphate treatment. The culture medium was changed every 2-3 days.

To investigate the effect of inorganic polyphosphate on native cartilage cultured ex vivo, 4 mm diameter cartilage samples were obtained from excised full thickness cartilage using a biopsy punch and cultured directly in 24-well plates under the same conditions as in vitro-formed cartilage.

### Histological Evaluation:

In vitro and ex vivo-cultured cartilage tissues were harvested and washed twice in PBS. The tissues were fixed in 10% formalin and embedded in paraffin. Cartilage formed on membrane inserts was processed with the membrane insert. Five micron sections were cut, stained with Toluidine blue and examined by light microscopy. To visualize the presence of polyphosphate in the tissues, sections were cut, dewaxed in xylene and stained with 4′-6-Diamidino-2-phenylindole (DAPI) (5 μg/ml, Pierce Biotechnology, Inc., Rockford, Ill., USA). The fluorescence was visualized with a Zeiss Axioplan epifluorescence microscope using a wide pass DAPI filter. Inorganic polyphosphate specifically shifts the emission peak of DAPI from 456 nm to 526 nm, permitting its visualization in the yellow-green spectrum rather than the blue spectrum associated with nucleic acids or glycosaminoglycans (GAG) [6,7].

### Dry Weight of Cartilaginous Tissue:

Cartilage tissues formed in vitro on membrane inserts were harvested, washed twice in PBS and removed from membrane inserts. The tissues were then lyophilized overnight and weighed using an electrical balance (Explorer, Ohaus Corp., Florham Park, N.J., USA).

### Determination of DNA Content

Cartilage tissues were digested with papain (Sigma Chemical Co., St. Louis, Mo., USA; 40 μg·ml⁻¹) in digestion buffer (20 mM ammonium acetate, 1 mM EDTA and 2 mM DTT) for 48 hours at 65° C. Papain digests then stored at −20° C. until further analysis. The DNA content was assessed using the Hoechst 33258 dye (Polysciences Inc., Washington, Pa., USA) binding assay and fluorometry (emission wavelength: 458 nm; excitation wavelength: 365 nm) [8]. The standard curve was generated with calf thymus DNA (Sigma Chemical Co., St. Louis, Mo., USA).

### Determination of Proteoglycan and Collagen Content:

The proteoglycan and collagen contents of cartilage tissues were also measured from aliquots of the papain digest. The proteoglycan content was estimated by quantifying the amount of sulphated GAG using the dimethylmethylene blue dye (Polysciences Inc., Washington, Pa., USA) binding assay and quantifying the colour spectrophotometrically at 525 nm [9]. The standard curve was generated with bovine trachea chondroitin sulphate A (Sigma Chemical Co., St. Louis, Mo., USA). The collagen content was estimated by quantifying the hydroxyproline. Papain digest aliquots were hydrolyzed in 6N HCl at 110° C. for 18 hours. The hydroxyproline of the hydrolysate was determined using the choramine-T/Ehrlich's reagent assay and the colour change quantified spectrophotometrically at 560 nm [10]. The standard curve was generated with L-hydroxyproline (Sigma Chemical Co., St. Louis, Mo., USA).

### Statistical Analysis:

All experiments were done in triplicate and repeated 3 times with cells from different extractions. Results are expressed as the mean±standard error of the mean unless specified otherwise and analyzed using a one-way ANOVA (for more than 2 conditions) or Student's t-test (between groups). Tukey's test post hoc analysis was performed. P values≦0.05 were considered statistically significant.

The results of the study are disclosed and discussed below.

### Inorganic Polyphosphate Stimulates Chondrocyte Matrix Accumulation

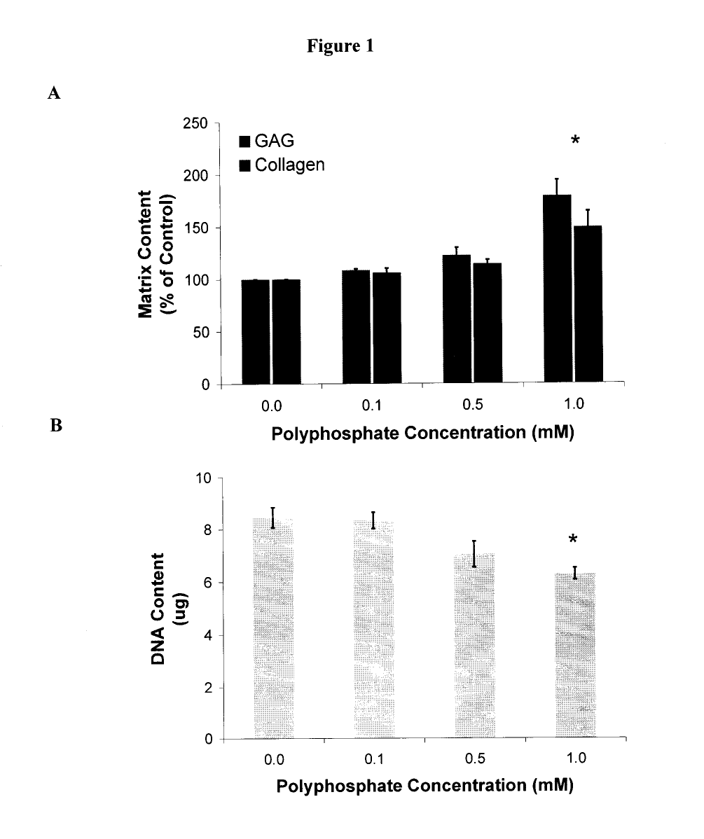

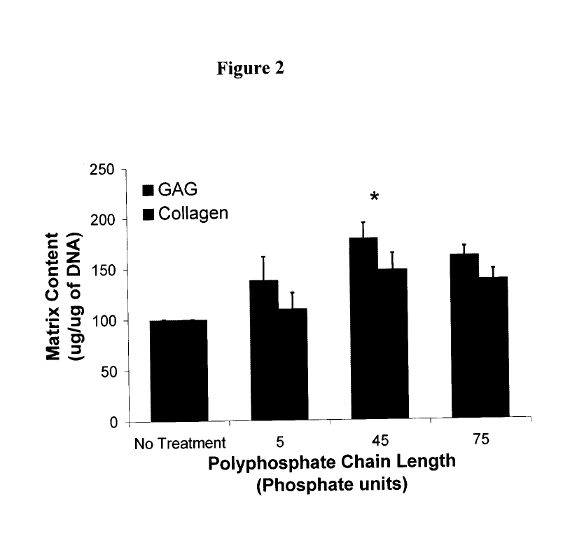

Full thickness chondrocytes were grown in vitro on membrane inserts for 2 weeks in media supplemented with different concentrations of inorganic polyphosphate (chain length of 45 phosphate units). Treatment with polyphosphate at a concentration of 1 mM resulted in significant increases in GAG and collagen accumulation per cell compared to cartilage formed in medium that was not supplemented with polyphosphate (FIG. 1A). Lower concentrations of polyphosphate resulted in more modest increases in matrix accumulation. The DNA content of in vitro-formed cartilage treated with 1 mM polyphosphate was also significantly lower than that in non-treated tissues (FIG. 1B).

Supplementation of the medium with equivalent concentrations of sodium phosphate to those used for polyphosphate treatment did not significantly alter the DNA, GAG and collagen contents in these tissues compared to cartilage formed in non-supplemented medium (Table I). This result suggests that the stimulatory effect of polyphosphate on cartilage matrix deposition is specific to condensed phosphate rather than its degradation product, orthophosphate.

### Effect of Inorganic Polyphosphate Chain Length on Matrix Accumulation

Chondrocytes were grown in vitro on membrane inserts for 2 weeks in medium supplemented with 1 mM inorganic polyphosphates characterized by various average chain lengths (5, 45 and 75 phosphate units). Polyphosphate with an average chain length of 45 phosphate units exhibited significant increases in both GAG and collagen contents in the tissues (FIG. 2). Based on these results, polyphosphate with an average chain length of 45 phosphate units was employed for all further studies.

### Time Course of Inorganic Polyphosphate Effect on Matrix Accumulation

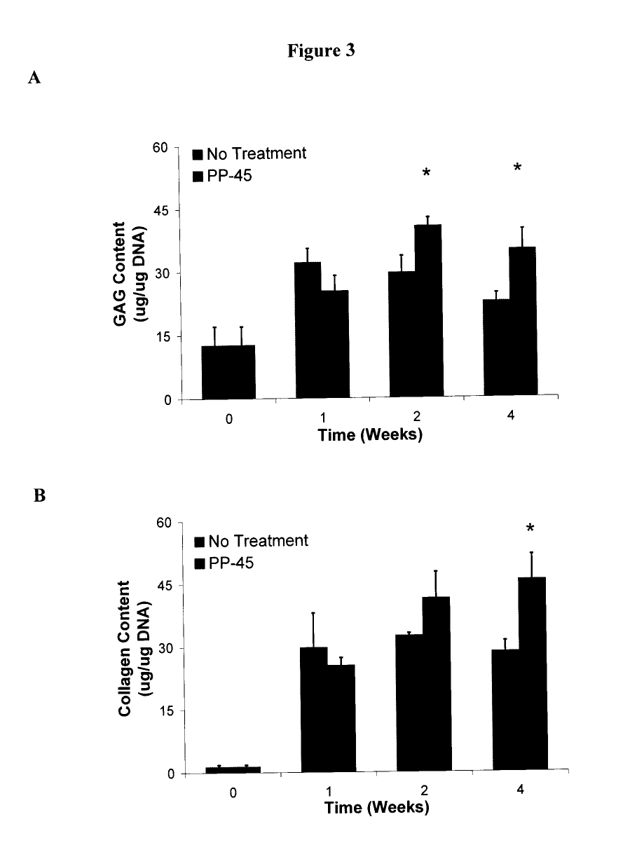

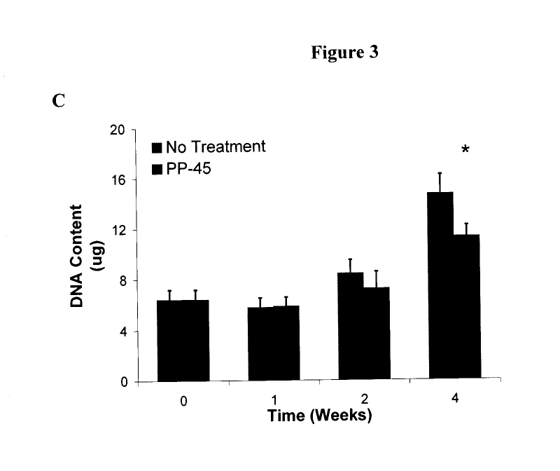

Chondrocytes were grown in vitro on membrane inserts for 0, 1, 2 or 4 weeks after initiation of the treatment with 1 mM polyphosphate in selected samples. The DNA content of non-treated tissues increased by more than 125% in 4 weeks, while polyphosphate treated tissues exhibited a more modest proliferation rate of 75% (FIG. 3C). The DNA content of treated tissues was significantly lower than that of controls at 4 weeks. After normalization by the DNA content, GAG (FIG. 3A) and collagen (FIG. 3B) accumulation within tissues formed in the absence of polyphosphate increases rapidly in the first week of culture and decreases by 4 weeks due to the increased tissue cellularity. Matrix accumulation within tissues treated with polyphosphate follows the same trend as in non-treated tissues but with significant increases in both GAG and collagen contents at 2 and 4 weeks of culture.

### The Stimulatory Effect of Inorganic Polyphosphate is Transient

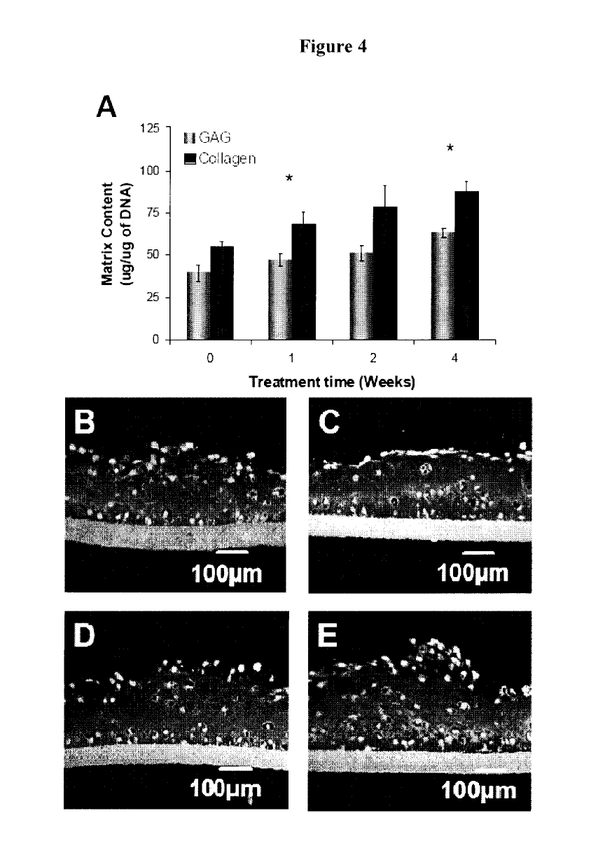

Chondrocytes were grown in vitro on membrane inserts for 4 weeks. The medium was supplemented with 1 mM polyphosphate for either the first 1 or 2 weeks of culture or for the entire 4 weeks period. The GAG content accumulated in the matrix of tissues stimulated with polyphosphate for parts of the entire incubation period were intermediate to that of non-treated controls and tissues treated for the entire 4 week period (FIG. 4A).

Epifluorescence imaging of DAPI stained tissue sections demonstrated that the polyphosphate accumulates within in vitro-formed cartilage treated with condensed phosphate for a period of 4 weeks (FIG. 4B). This accumulation was less obvious upon interruption of the treatment (FIG. 4C-D). In contrast, non-treated cartilage exhibits the lowest levels of polyphosphate (FIG. 4E).

Deep-Zone Chondrocytes are More Responsive than Superficial-Mid Zone Chondrocytes to Inorganic Polyphosphate

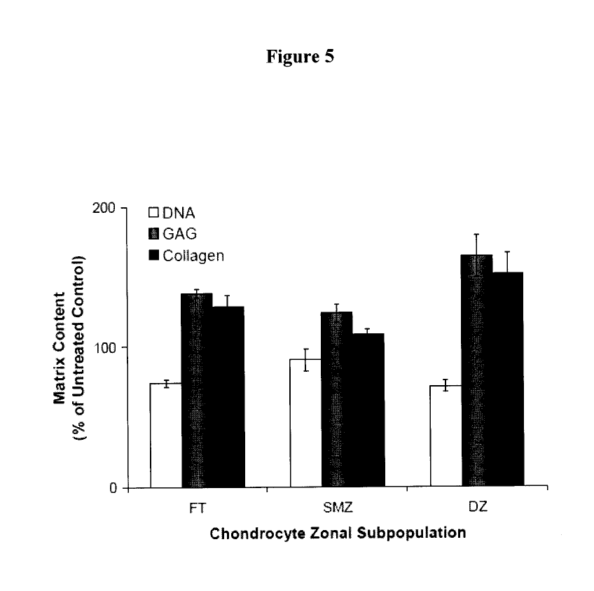

Two chondrocyte subpopulations (superficial-mid zone and deep zone) were grown in vitro on membrane inserts for 2 weeks in medium supplemented with 1 mM inorganic polyphosphates and compared to zonal sub-population tissues formed without supplementation and full thickness chondrocytes. FIG. 5 shows the DNA, GAG and collagen contents of tissues formed by treatment of the different chondrocyte subpopulations with 1 mM polyphosphate as a percentage of the contents in untreated tissues. No significant difference was observed in the responsiveness of the different subpopulations to polyphosphate, but a trend towards a larger effect in deep zone cell cultures were observed for the three parameters measured (DNA, GAG and collagen contents).

### Inorganic Polyphosphate Stimulates Matrix Accumulation in Native Cartilage

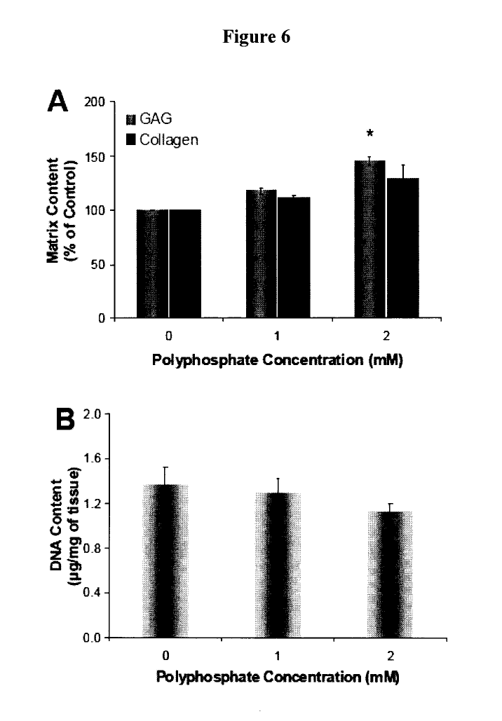

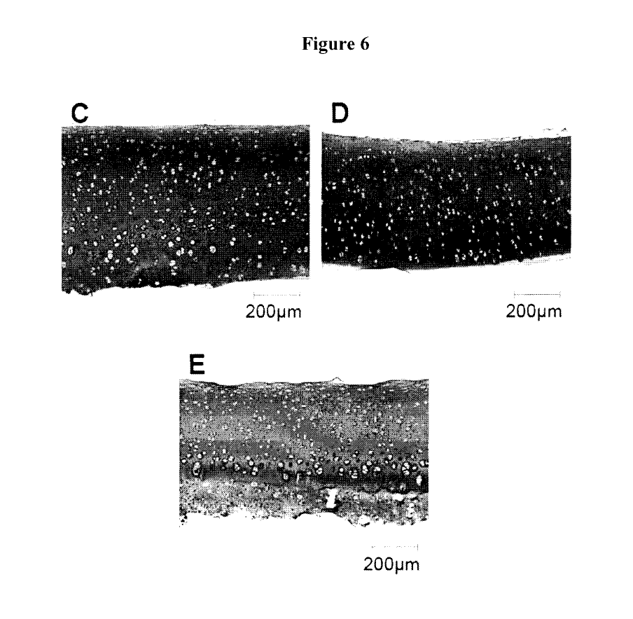

Native cartilage samples were obtained from excised full thickness cartilage and cultured ex vivo directly on tissue culture plates for 1 week and selected samples were treated with polyphosphate at concentrations of 1 or 2 mM. This study was performed to verify that the anabolic effects of polyphosphate were not limited to immature in vitro-formed cartilage. As is shown in FIG. 6A, the GAG content of native cartilage samples was significantly increased by approximately 45% following supplementation of the culture medium with polyphosphate. The collagen content was also increased by approximately 30% but this difference was not significant. The DNA content of cartilage samples was not significantly decreased by treatment with polyphosphate (FIG. 6B).

A histological evaluation of ex vivo-cultured native cartilage samples stained with Toluidine blue shows extensive new matrix deposited around tissues stimulated with polyphosphate compared to non-treated cartilage (FIGS. 6C, 6D and 6E). This matrix was deposited predominantly on the deep-zone face of cartilage samples. It is not possible to determine if increased matrix deposition occurred within the native tissue as well.

This study demonstrates that inorganic polyphosphate exogenously administered to bioengineered cartilage during the in vitro maturation phase or to native articular cartilage cultured ex vivo promotes GAG and collagen accumulation in the extracellular matrix. The anabolic effect of polyphosphate on in vitro-formed cartilage matrix deposition was concentration and chain length dependent. Conversely, polyphosphate treatment resulted in a lower increase in DNA content of bioengineered cartilage compared to untreated tissues. The continuous presence of condensed phosphate in culture is essential to the full stimulatory effects on matrix accumulation, as full thickness cartilage expresses exopolyphosphatases that degrade polyphosphate. The effect of polyphosphate on matrix accumulation appears to be slightly more pronounced on deep-zone chondrocytes than on superficial-mid zone mixed-population chondrocytes. These results suggest that inorganic polyphosphate can be used to stimulate the production of cartilage extracellular matrix components in bioengineered cartilage.

This is the first report of the stimulatory effect of inorganic polyphosphate on articular cartilage matrix accumulation. It is possible that condensed phosphates could stabilize endogenous growth factors such as FGF-2 physically and functionally as demonstrated by Shiba et al. (2003) [11]. Treatment of in vitro-formed cartilage with inorganic polyphosphate leads to the upregulation of cartilage matrix genes such as aggrecan and collagen type II, while not affecting the expression of collagen type X and type I, suggesting that polyphosphate stabilizes the chondrocytes phenotype.

Increases in GAG and collagen contents were observed in cartilage tissues treated with polyphosphates of average chain length of 5, 45 and 75 phosphate units.

Interestingly, treatment with inorganic polyphosphate resulted in an increase in DNA content of the in vitro-formed cartilage over time; albeit at a much slower rate than in non-treated tissues. This result suggests that polyphosphate acts to inhibit chondrocyte proliferation in the system studied. However, polyphosphate has been shown to stimulate proliferation in human fibroblasts and a murine pre-osteoblastic cell line through the FGF signalling pathway [11] as well as in mammary cancer cells through the mTor signalling pathway [12]. This discrepancy may be due to cell type specific effects.

The extent of cartilage matrix accumulation is directly related to the percentage of the culture period in which the bioengineered tissues are exposed to inorganic polyphosphate. The action of exopolyphosphatases with the ability to cleave phosphoanhydride bonds of the condensed phosphates along with hydrolysis can lead to a rapid loss of the effects of polyphosphate [13,14,15]. In this study, polyphosphate accumulation was much more prominent in tissues which were treated with the condensed polymers for the entire culture period than in tissues which received treatment for only parts of that period corroborating these results.

Polyphosphate treatment of native cartilage samples ex vivo showed increased GAG and collagen contents compared to untreated controls, supporting results obtained with bioengineered cartilage. Histological appearance of native cartilage tissues cultured ex vivo shows that new matrix accumulation was deposited predominantly on the deep zone cartilage. This is corroborated by the increased response of deep-zone chondrocytes to polyphosphate treatment in vitro compared to superficial-mid zone chondrocytes. The lack of effect of polyphosphate on superficial-mid zone tissue DNA content might be explained by a lower proliferation potential than deep-zone chondrocytes. However, the fainter effect of polyphosphate on GAG and collagen accumulation within the superficial-mid zone cartilage suggests an increased responsiveness of deep zone chondrocytes to the treatment. The differential response of chondrocyte sub-populations to inorganic polyphosphate may provide a useful system to understand the pathways by which condensed phosphate stimulates cartilage matrix deposition.

In summary, this study demonstrates that inorganic polyphosphate stimulates cartilage matrix accumulation in both in vitro-formed cartilage and ex vivo cultured native cartilage. Based on these results, polyphosphate treatment may be useful to improve the quality of in vitro-formed bioengineered cartilage.

### Example 2

### Intra-Articular Injections of Polyphosphate (PP) Prevented Disease Progression in a Guinea Pig Osteoarthritis Model

The following materials and methods were used in the study described in this Example.

Animal Model:

3 month male Hartley guinea pigs were randomly divided in 2 groups (n=10/group). [16] Medial menisectomy was performed on the right leg to ensure early and predictable progression of osteoarthritis [17, 18]. The animals received intra-articular injections (2 times/wk) of either PP (4 mM in PBS, average chain length of 45 phosphate units) or PBS (carrier) (30 μl) starting one day after the surgery. The polyphosphate concentration was determined based on the weight of the polyphosphate. The animals were sacrificed 2 months post-operatively and the knee joint (stifle) harvested. The effect of treatment on OA progression was evaluated. The gross appearance of the cartilage was graded using the ICRS scale. [19] The tibial plateau was imaged by microCT. Coronal sections of the tibial plateau were cut to include the articular surface and subchondral bone.

Decalcified Bone Sections:

The tibial plateau was fixed in 10% buffered formalin, decalcified in 0.5 mM EDTA, and embedded in paraffin. 5 μm sections were stained with toluidine blue or hematoxylin and eosin. The histological changes in the cartilage were graded using a grading system developed for the guinea pig. [20]

Cartilage Biochemistry:

Articular cartilage was collected from the femoral condyle. The tissue was papain digested (40 μg/ml in 20 mM ammonium acetate, 1 mM EDTA, and 2 mM dithiotreitol) for 48 h at 65° C. From these digests the DNA content was determined using the Hoechst 33258 dye binding assay (Polysciences, PA) and fluorometry. The proteoglycan content was determined by quantifying sulphated glycosaminoglycans using the dimethylmethylene blue dye binding assay and spectrophotometry. Collagen content was determined by quantifying OH-proline levels after HCl hydrolysis of the papain digest using the chloramine-T/Ehlrich's reagent dye binding assay and spectrophotometry. All samples were assayed in triplicate.

### Statistical Analysis:

Data is presented as the mean±SEM. The data was analyzed using the Mann-Whitney Rank Sum Test or two-way ANOVA. Significance was assigned at p<0.05.

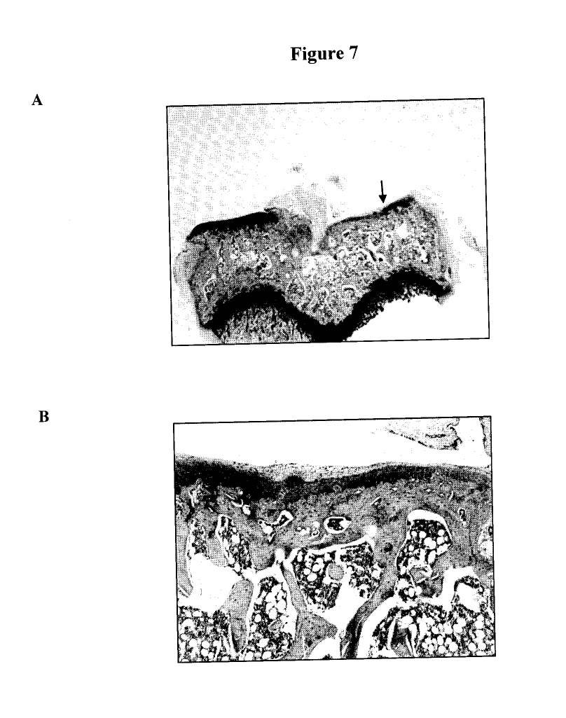

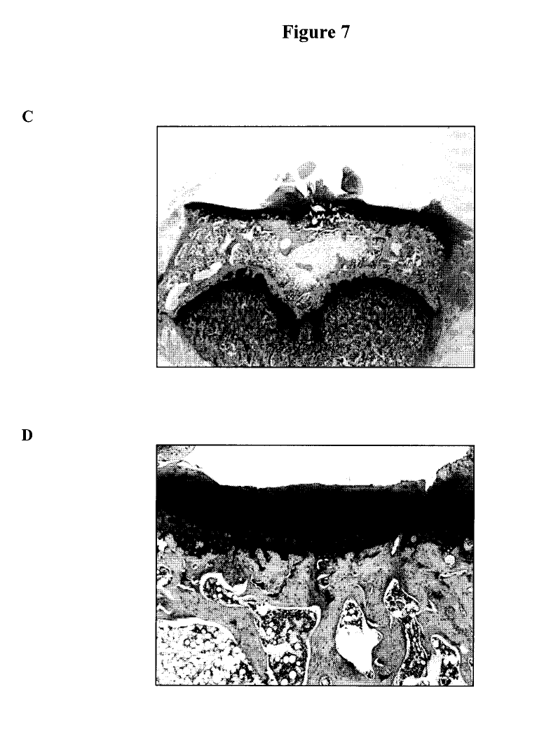

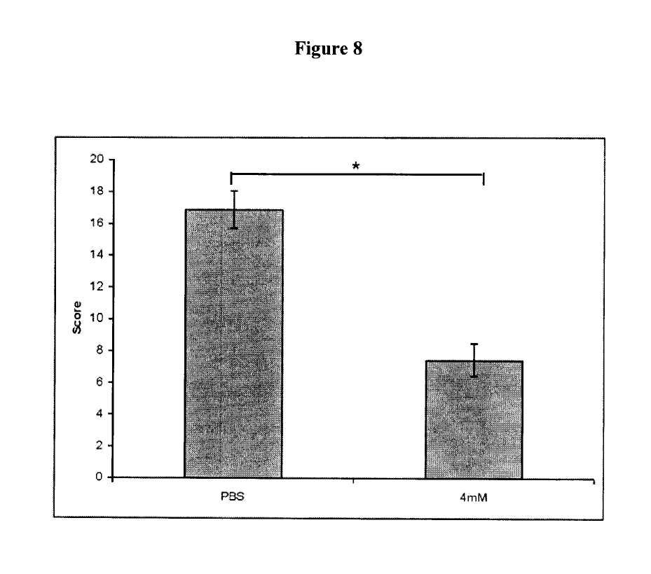

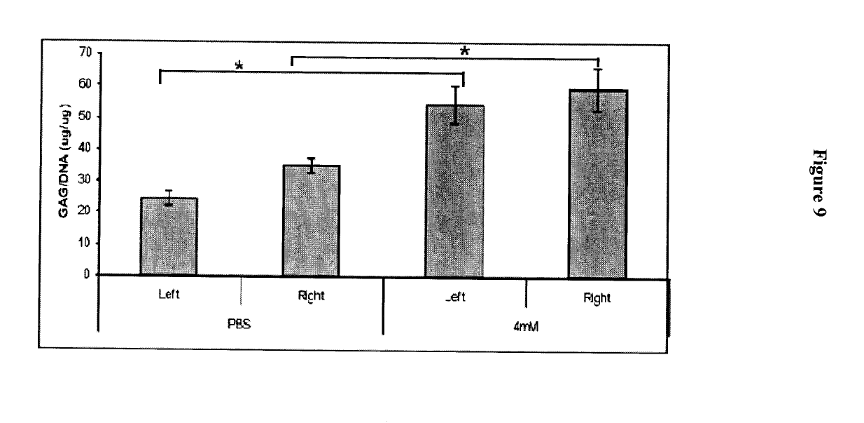

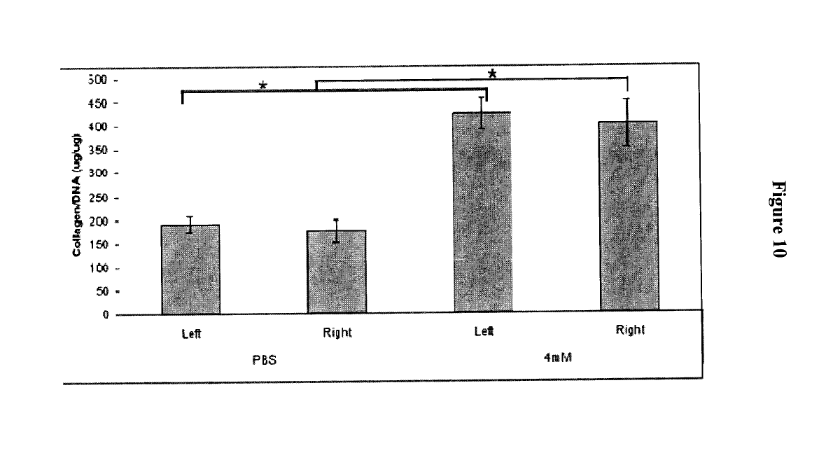

The results of the study are as follows. The animals in all groups showed evidence of osteoarthritis in the operated knee. Treatment appeared to decrease the extent of osteoarthritic changes (FIG. 7). There was significantly less degenerative changes (p<00.1) in the PP treated guinea pigs as compared to the control group. The histological grade of the degenerative changes in the tibial plateau in the control animals was 16.9±1.2 (mean±SEM) whereas the polyphosphate treated animals (4 mM) had an average score of 7.4±1.0 (FIG. 8). The biochemical analysis indicated a significant increase in collagen and proteoglycan contents normalized to DNA content in the PP treated as compared to the control guinea pigs suggesting the presence of more tissue (FIGS. 9 and 10). Interestingly the amount of extracellular matrix (tissue) was also increased on the PP treated joints on the side of the joint that did not experience any surgical intervention.

### Example 3

### Preparation of a Short Chain Polyphosphate

The first step in the preparation of a short chain polyphosphate is the calcining of the precursor powder, phosphate monobasic monohydrate (Ca(H₂PO₄)₂.H₂O, CPPM (J T Baker, Phillipsburg, N.J.). Approximately 70 g of the powder was placed in a 125 cm³ platinum crucible and calcined at 500° C. for 10 hours in air resulting in calcium polyphosphate formation through the following condensation reaction:

n(Ca(H₂PO₄)₂.H₂O→[Ca(PO₃)₂]ₙ+3nH₂O

The resulting powder was melted at 1100° C. to produce an amorphous glass and held for 1 hour at the same temperature. Molten calcium polyphosphate was poured directly into distilled water, which due to the rapid cooling resulted in the formation of an amorphous fit. The fit was dried in 100% ethanol to remove absorbed water and subsequently stored in a vacuum desiccator until further processing. Powder of the desired size was produced from the frit by ball milling using a stainless steel mortar and balls followed by screening. Powders having a size range of between 75-150 μm were selected and dissolved in a selected solvent (e.g. a buffer such as water, PBS or media) and 3 hours while mixing. The solution is then filtered and the polyphosphate concentration is measured enzymatically (e.g., see Example 4) to confirm the concentration.

### Example 4

Inorganic polyphosphate concentration can be measured enzymatically using the following method. Briefly, an aliquot of the polyphosphate solution is incubated at room temperature with calf intestinal alkaline phosphatase (1 unit per reaction) in 50 mM Tris buffer (pH 9.4) for 30 minutes. The phosphate level is then measured by reaction with a solution containing 1:6 v/v ratio of 10% ascorbic acid and 0.42% ammonium molybdate in 1N H₂SO₄ (1:3 v/v ratio) at 37° C. for 1 hour and the resulting color quantified by spectrophotometrical analysis at 620 nm. A standard curve was generated using a sodium phosphate dibasic solution (pH=7.5). The polyphosphate content (on a phosphate basis) is calculated by subtracting the phosphate content of aliquots not digested with alkaline phosphatase to that in enzymatically digested aliquots.

### Example 5

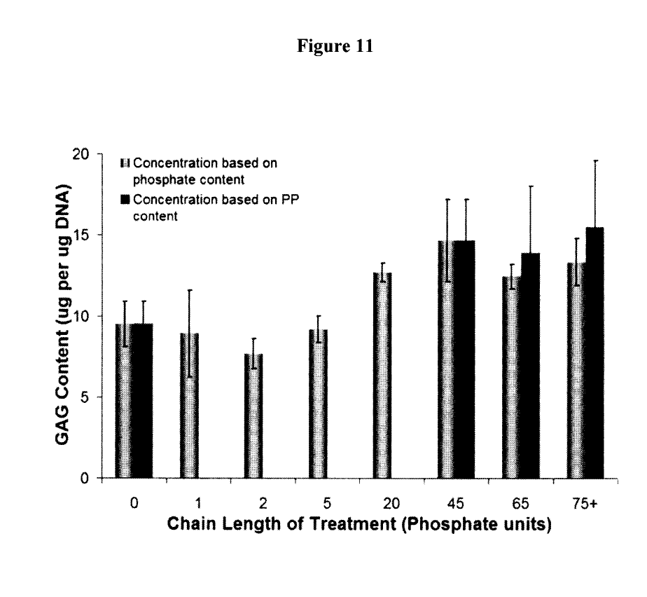

In vitro cultured cartilage was treated with 0.5 mM (enzymatically determined as in Example 4) polyphosphate of various chain lengths in DMEM+20% FBS. Cultures were grown for 10 days following initiation of the polyphosphate treatment and GAG and DNA content were assayed (see Example 1). The concentration of GAG content normalized to DNA content is shown in FIG. 11.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the methodologies etc. which are reported therein which might be used in connection with the invention. Nothing herein is to be construed as an admission that the invention is not entitled to antedate such disclosure by virtue of prior invention.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content short chain polyphosphate includes a mixture of two or more short chain polyphosphates.

TABLE I
Glycosaminoglycan and Collagen Contents of Cartilage

| | DNA Content | GAG Content | Collagen Content |
|---|---|---|---|
| Treatment | (μg) | (% of control) | (% of control) |
| None | 9.05 ± 0.25 | 100.0 ± 0.0 | 100.0 ± 0.0 |
| Phosphate | 8.5 ± 0.92 | 112.5 ± 14.8 | 105.3 ± 9.5 |
| Polyphosphate | 6.8 ± 0.71 | 178.9 ± 15.1* | 147.6 ± 16.1* |

Chondrocytes were grown in the presence of phosphate (1 mM) or polyphosphate (1 mM based on the phosphate content; chain length of 45 phosphate units).
The DNA, GAG and Collagen contents were determined after 2 weeks in culture. The data is averaged from three experiments performed in triplicates and expressed as mean ± standard error of the mean. The matrix content is normalized to DNA content and matrix content of untreated controls.
*indicates a significantly different content than untreated tissues (p < 0.05).

### FULL CITATIONS FOR REFERENCES

[1] Hunziker E B. Articular cartilage repair: are the intrinsic biological constraints undermining this process insuperable? Osteoarthritis Cartilage 1999; 7(1):15-28.
[2] Kinner B, Capito R M, Spector M. Regeneration of articular cartilage. Adv Biochem Eng Biotechnol 2005; 94:91-123.
[3] Buschmann M D, Gluzband Y A, Grodzinsky A J, Hunziker E B. Mechanical compression modulates matrix biosynthesis in chondrocyte/agarose culture. J Cell Sci 1995; 108 (Pt 4):1497-508.
[4] Waldman S D, Grynpas M D, Pilliar R M, Kandel R A. Characterization of cartilagenous tissue formed on calcium polyphosphate substrates in vitro. J Biomed Mater Res 2002; 62(3):323-30.
[5] Yu H, Grynpas M, Kandel R A. Composition of cartilagenous tissue with mineralized and non-mineralized zones formed in vitro. Biomaterials 1997; 18(21):1425-31.
[6] Allan R A, Miller J J. Influence of S-adenosylmethionine on DAPI-induced fluorescence of polyphosphate in the yeast vacuole. Can J Microbiol 1980; 26(8):912-20.
[7] Tijssen J P, Beekes H W, Van Steveninck J. Localization of polyphosphates in Saccharomyces fragilis, as revealed by 4′,6-diamidino-2-phenylindole fluorescence. Biochim Biophys Acta 1982; 721(4):394-8.
[8] Kim Y J, Sah R L, Doong J Y, Grodzinsky A J. Fluorometric assay of DNA in cartilage explants using Hoechst 33258. Anal Biochem 1988; 174(1):168-76.
[9] Goldberg R L, Kolibas L M. An improved method for determining proteoglycans synthesized by chondrocytes in culture. Connect Tissue Res 1990; 24(3-4):265-75.
[10] Reddy G K, Enwemeka C S. A simplified method for the analysis of hydroxyproline in biological tissues. Clin Biochem 1996; 29(3):225-9.
[11] Shiba T, Nishimura D, Kawazoe Y, Onodera Y, Tsutsumi K, Nakamura R, et al. Modulation of mitogenic activity of fibroblast growth factors by inorganic polyphosphate. J Biol Chem 2003; 278(29):26788-92.
[12] Wang L, Fraley C D, Faridi J, Kornberg A, Roth R A. Inorganic polyphosphate stimulates mammalian TOR, a kinase involved in the proliferation of mammary cancer cells. Proc Natl Acad Sci USA 2003; 100(20):11249-54.
[13] St-Pierre J P, Pilliar R M, Grynpas M D, Kandel R A. Calcification of cartilage formed in vitro on calcium polyphosphate bone substitutes is regulated by inorganic polyphosphate. Acta Biomater 2010.
[14] Fleisch H, Neuman W F. Mechanisms of calcification: role of collagen, polyphosphates, and phosphatase. Am J Physiol 1961; 200(6):1296-300.
[15] Francis M D. The inhibition of calcium hydroxypatite crystal growth by polyphosphonates and polyphosphates. Calcif Tissue Res 1969; 3(2):151-62.
[16] Kraus V B, Huebner J L, DeGroot J, Bendele A. The OARSI histopathology initiative—recommendations for histological assessments of osteoarthritis in the guinea pig. Osteoarthritis Cartilage. 2010 October; 18 Suppl 3:S35-52.
[17] Bendele A M, White S I, Hulman J F, Bean J S. Osteoarthritis, Model No. 389. In: Capen, C C, Jones T C, Migaki (eds) Handbook: Animal models of human disease. Fasc. 18. Registry of comparative pathology, Washington D.C., 1991.
[18] Bendele A M. Animal Models of osteoarthritis in an era of molecular biology. 2002. J Musculoskel Neuron Interact 2(6): 501-503.
[19] Brittberg M and Winalski C S. Evaluation of Cartilage Injuries and Repair. 2003 J Bone Joint Surg Am. 85:58-69; ICRS grading: http://www.cartilage.org/_files/contentmanagement/ICRS_evaluation.pdf
[20] Kraus V B, Huebner J L, DeGroot J, Bendele A. The OARSI histopathology initiative—recommendations for histological assessments of osteoarthritis in the guinea pig. Osteoarthritis Cartilage. 2010 October; 18 Suppl 3:S35-52.

1. A method of stimulating cartilage growth, regeneration or repair in a subject where cartilage growth, regeneration or repair is desired comprising administering to the subject a therapeutically effective amount of a short chain polyphosphate, wherein the short chain polyphosphate has greater than 5 phosphate units.

2. A method of claim 1 wherein the subject has osteoarthritis.

3. A method of claim 1, wherein a therapeutically effective amount of short chain polyphosphate effective for reducing the progression of cartilage destruction or restoring or increasing cartilage matrix is administered periodically.

4. A method of claim 1, wherein a therapeutically effective amount of short chain polyphosphate effective for reducing the progression of cartilage destruction or restoring or increasing cartilage matrix is continuously administered.

5. A method of claim 1, wherein the polyphosphate is administered by intra-articular injection, subcutaneous injection or oral administration.

6. A method of claim 1, wherein the polyphosphate is administered at a site of injury in the subject.

7. A method of claim 1, wherein the short chain polyphosphate has an average chain length of 45 phosphate units.

8. A method of claim 1, wherein the therapeutically effective amount of polyphosphate ranges from about 10 ng to 100 mg phosphate.

9. A method of claim 1, wherein the therapeutically effective amount of polyphosphate is between about 0.01 mg and 20 mg phosphate.

10. A composition for the treatment of a cartilage disease comprising an amount of a short chain polyphosphate effective for reducing the progression of cartilage destruction or restoring or increasing cartilage matrix and a pharmaceutically acceptable carrier, excipient or vehicle, wherein the short chain polyphosphate has greater than 5 phosphate units.

11. A composition as claimed in claim 10, wherein the pharmaceutically acceptable carrier, excipient or vehicle is of a type suitable for the formulation of the composition for intra-articular or subcutaneous injection, or oral administration.

12. A composition as claimed in claim 10, wherein the amount of polyphosphate ranges from about 0.01 mg to 20 mg phosphate.

13. (canceled)

14. A method of stimulating production of cartilage extracellular matrix components in bioengineered cartilage or ex vivo cultured cartilage or of promoting glycosaminoglycans (GAG) and collagen accumulation in the extracellular matrix of in vitro formed cartilage or ex vivo cultured cartilage comprising culturing the cartilage in the presence of a short chain polyphosphate, wherein the short chain polyphosphate has greater than 5 phosphate units.

15-16. (canceled)
